(19) United States
(12) Patent Application Publication
(10) Pub. No.: US 2025/0241917 A1
(43) Pub. Date: Jul. 31, 2025

(54) SMALL MOLECULE ENHANCERS OF ANTISENSE OLIGO ACTIVITY

(71) Applicant: Oregon State University, Corvallis, OR (US)

(72) Inventors: Hong M. Moulton, Corvallis, OR (US); Scott David Bittner, Corvallis, OR (US)

(73) Assignee: Oregon State University, Corvallis, OR (US)

(21) Appl. No.: 18/856,043

(22) PCT Filed: Apr. 12, 2023
(86) PCT No.: PCT/US2023/018374

§ 371 Date: Oct. 10, 2024

Related U.S. Application Data
(60) Provisional application No. 63/330,583, filed on Apr. 13, 2022.

(51) Int. Cl. A61K 31/519; A61K 31/05; A61K 31/426; A61K 31/4545; A61K 45/06

(52) U.S. Cl. CPC A61K 31/519; A61K 31/05; A61K 31/426; A61K 31/4545; A61K 45/06

(57) ABSTRACT

Methods and compositions for the cytosolic delivery of bioactive macromolecules. In the methods, an effective amount of an enhancer molecule is used to facilitate the cytosolic delivery of the bioactive macromolecule.

### CROSS-REFERENCE TO RELATED APPLICATION

The present application claims the benefit of U.S. Application No. 63/330,583, filed Apr. 13, 2022, expressly incorporated herein by reference in its entirety.

### BACKGROUND

Bioactive macromolecules such as antisense oligos, RNA, siRNA, peptides and proteins all hold great therapeutic promise. However, the therapeutic use of such macromolecules is often hampered by limited delivery into the cytosol of cells or limited cellular uptake.

A need exists for improving cytosolic delivery of bioactive macromolecules to advance the use of such macromolecules as therapeutic agents. The present disclosure seeks to fulfill this need and provides further related advantages.

### SUMMARY

The present disclosure provides methods and compositions for cytosolic delivery of bioactive macromolecules.

In one aspect, the disclosure provides a method for cytosolic delivery of bioactive macromolecules in a subject. In one embodiment, the method comprises administering to a subject in need thereof a therapeutically effective amount of a bioactive macromolecule and an effective amount of an enhancer molecule selected from the group consisting of febuxostat, lonafarnib, nitazoxanide, CGS-15943, and resveratrol and pharmaceutically acceptable salts thereof.

Representative bioactive macromolecules delivered in the methods include antisense oligos, RNAs, siRNAs, peptides, and proteins. In certain embodiments, the bioactive macromolecule is an antisense oligo.

Representative antisense oligos delivered in the methods includes phosphorodiamidate morpholino oligomers (PMOs), peptide-conjugated phosphorodiamidate morpholino oligomers (PPMOs), 2′-O-methyl phosphorothioate (2′OMe PS) oligomers, 2′-O-methoxyethyl phosphorothioate (2′-MOE PS) oligomers, peptide nucleic acids (PNA), locked nucleic acids (LNA), LNA and 2′OMe PS gapmers, LNA and 2′MOE gapmers, gapmers of 2′OMe PS and natural nucleic acids, gapmers of 2′OMOE PS and natural nucleic acids, and unmodified or modified nucleic acids. In certain embodiments, the antisense oligo is a phosphorodiamidate morpholino oligomer (PMO), a peptide-conjugated phosphorodiamidate morpholino oligomer (PPMO), a 2′-O-methyl phosphorothioate (2′OMe PS) oligomer, or a 2′-O-methoxyethyl phosphorothioate (2′-MOE PS) oligomer.

In a related embodiment, the present disclosure provides a method for intracellular delivery of antisense oligo. In certain of these embodiments, the method provides a method for intracellular antisense oligo delivery, comprising contacting a cell with an antisense oligo, and an effective amount of an enhancer molecule selected from the group consisting of febuxostat, lonafarnib, nitazoxanide, CGS-15943, and resveratrol, and pharmaceutically acceptable salts thereof. In another of these embodiments, the method provides a method for intracellular antisense oligo delivery to a subject, comprising administering to a subject in need thereof an antisense oligo, and an effective amount of an enhancer molecule selected from the group consisting of febuxostat, lonafarnib, nitazoxanide, CGS-15943, and resveratrol, and pharmaceutically acceptable salts thereof.

In certain of the above embodiments, the antisense oligo is phosphorodiamidate morpholino oligomer (PMO), a peptide-conjugated phosphorodiamidate morpholino oligomer (PPMO), a 2′-O-methyl phosphorothioate (2′OMe PS) oligomer, a 2′-O-methoxyethyl phosphorothioate (2′-MOE PS) oligomer, a peptide nucleic acids (PNA), a locked nucleic acid (LNA), a LNA or a 2′OMe PS gapmer, a LNA or 2′MOE gapmer, a gapmer of 2′OMe PS or a natural nucleic acid, a gapmer of 2′OMOE PS or a natural nucleic acid, or unmodified or modified nucleic acid.

In certain of these embodiments, the antisense oligo is a phosphorodiamidate morpholino oligomer (PMO), a peptide-conjugated phosphorodiamidate morpholino oligomer (PPMO), a 2′-O-methyl phosphorothioate (2′OMe PS) oligomer, or a 2′-O-methoxyethyl phosphorothioate (2′-MOE PS) oligomer.

In another aspect, the present disclosure provides a composition for delivery of a bioactive macromolecule. In certain embodiments, the composition comprises a bioactive macromolecule and an enhancer molecule selected from the group consisting of febuxostat, lonafarnib, nitazoxanide, CGS-15943, and resveratrol, and pharmaceutically acceptable salts thereof. In certain embodiments, the enhancer molecule is febuxostat or a pharmaceutically acceptable salt thereof.

In certain embodiments, the bioactive macromolecule is an antisense oligo, an RNA, an siRNA, a peptide, or a protein. In certain of these embodiments, the bioactive macromolecule is an antisense oligo. Representative antisense oligos include phosphorodiamidate morpholino oligomers (PMOs), peptide-conjugated phosphorodiamidate morpholino oligomers (PPMOs), 2′-O-methyl phosphorothioate (2′OMe PS) oligomers, 2′-O-methoxyethyl phosphorothioate (2′-MOE PS) oligomers, peptide nucleic acids (PNA), locked nucleic acids (LNA), LNA and 2′OMe PS gapmers, LNA and 2′MOE gapmers, gapmers of 2′OMe PS and natural nucleic acids, gapmers of 2′OMOE PS and natural nucleic acids, and unmodified or modified nucleic acids. In certain embodiments, the antisense oligo is a phosphorodiamidate morpholino oligomer (PMO), a peptide-conjugated phosphorodiamidate morpholino oligomer (PPMO), a 2′-O-methyl phosphorothioate (2′OMe PS) oligomer, or a 2′-O-methoxyethyl phosphorothioate (2′-MOE PS) oligomer.

In other embodiments, the present disclosure provides a composition comprising an antisense oligo and an enhancer molecule selected from the group consisting of febuxostat, lonafarnib, nitazoxanide, CGS-15943, and resveratrol, and pharmaceutically acceptable salts thereof.

In a further aspect, the present disclosure provides methods for treating a disease or condition treatable by administering an antisense oligo. In certain embodiments, the methods comprise administering to a subject in need thereof a therapeutically effective amount of an antisense oligo useful in treating a disease or condition and an effective amount of an enhancer molecule. In these methods, the enhancer molecule is selected from febuxostat, lonafarnib, nitazoxanide, CGS-15943, and resveratrol, and pharmaceutically acceptable salts thereof.

The present disclosure provides the use of a composition as described herein for cytosolic delivery of a bioactive macromolecule to a subject, for intracellular delivery of an antisense oligo or an RNA to a subject, for treating a disease or condition treatable by administering an antisense oligo, and for improving the cytosolic delivery of a bioactive macromolecule in a subject, animals, and cells.

The present disclosure provides methods for improving the cytosolic delivery of bioactive macromolecules in a human subject, animal, or cell, comprising administering to the cell an enhancer molecule sufficient to increase the cytosolic delivery of the bioactive macromolecule.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 shows antisense oligo (ASO) activity (RLU) as a function of a representative oligo activity enhancer (OAE) (febuxostat) concentration (μM) (secondary screen: PPMO concentration fixed at 5 μM, enhancer concentration varied). Mean of four replicates shown (eight replicates for PPMO only), error bars represent standard deviation. RLU=relative light unit.

FIG. 2 shows antisense oligo (ASO) activity (RLU) as a function of a representative oligo activity enhancer (OAE) (lonafarnib) concentration (μM) (secondary screen: PPMO concentration fixed at 5 μM, enhancer concentration varied). Mean of four replicates shown (eight replicates for PPMO only), error bars represent standard deviation. RLU=relative light unit.

FIG. 3 shows antisense oligo (ASO) activity (RLU) as a function of a representative oligo activity enhancer (OAE) (resveratrol) concentration (μM) (secondary screen: PPMO concentration fixed at 5 μM, enhancer concentration varied). Mean of four replicates shown (eight replicates for PPMO only), error bars represent standard deviation. RLU=relative light unit.

FIG. 4 shows antisense oligo (ASO) activity (RLU) as a function of a representative oligo activity enhancer (OAE) (nitazoxanide) concentration (μM) (secondary screen: PPMO concentration fixed at 5 μM, enhancer concentration varied). Mean of four replicates shown (eight replicates for PPMO only), error bars represent standard deviation.

FIG. 5 shows antisense oligo (ASO) activity (RLU) as a function of a representative oligo activity enhancer (OAE) (CGS-15943) concentration (p M) (secondary screen: PPMO concentration fixed at 5 μM, enhancer concentration varied). Mean of four replicates shown (eight replicates for PPMO only), error bars represent standard deviation.

FIGS. 6A-6C compare antisense oligo (ASO) activity (RLU) as a function of a representative oligo activity enhancer (OAE) (febuxostat) concentration (μM) in an HeLa pLuc/705 assay (varied OAE concentration with fixed ASO concentration: PPMO at 5 μM (6A); 2′-OMe PS at 5 μM (6B); and 2′-MOE PS at 2.5 μM (6C). Mean of four replicates shown, error bars represent standard deviation.

FIGS. 7A-7C compare antisense oligo (ASO) activity (RLU) as a function of a representative oligo activity enhancer (OAE) (lonafarnib) concentration (μM) in an HeLa pLuc/705 assay (varied OAE concentration with fixed ASO concentration: PPMO at 5 μM (7A); 2′-OMe PS at 5 μM (7B); and 2′-MOE PS at 2.5 μM (7C). Mean of four replicates shown, error bars represent standard deviation.

FIGS. 8A-8C compare antisense oligo (ASO) activity (RLU) as a function of a representative oligo activity enhancer (OAE) (CGS-15943) concentration (μM) in an HeLa pLuc/705 assay (varied OAE concentration with fixed ASO concentration: PPMO at 5 μM (8A); 2′-OMe PS at 5 μM (8B); and 2′-MOE PS at 2.5 μM (8C). Mean of four replicates shown, error bars represent standard deviation.

FIGS. 9A and 9B compare antisense oligo (ASO) activity (RLU) as a function of a representative oligo activity enhancer (OAE) (resveratrol) concentration (μM) in an HeLa pLuc/705 assay (varied OAE concentration with fixed ASO concentration: PPMO at 5 μM (9A); and 2′-OMe PS at 5 μM (9B). Mean of four replicates shown, error bars represent standard deviation.

FIG. 10 shows antisense oligo (ASO) activity (RLU) as a function of ASO (PMO) concentration (2.5 μM) with fixed oligo activity enhancer (OAE) (febuxostat) concentration (μM) in an HeLa pLuc/705 assay (varied PMO concentration, enhancer concentration fixed at 2.5 μM). Mean of three replicates shown, error bars represent standard deviation. For each set of bars, the result with PMO is on the left and PMO plus febuxostat is on the right.

FIG. 11 shows antisense oligo (ASO) activity (RLU) as a function of ASO (PMO) concentration (μM) with fixed oligo activity enhancer (OAE) (lonafarnib) concentration (250 nM) in an HeLa pLuc/705 assay (varied PMO concentration, enhancer concentration fixed at 250 nM). Mean of three replicates shown, error bars represent standard deviation. For each set of bars, the result with PMO is on the left and PMO plus lonafarnib is on the right.

FIG. 12 shows antisense oligo (ASO) activity (RLU) as a function of ASO (2′-OMe phosphorothioate, 2′-OMe PS) concentration (μM) with fixed oligo activity enhancer (OAE) concentration in an HeLa pLuc/705 assay (varied ASO concentration, enhancer concentrations fixed: febuxostat, 5 μM; lonafarnib, 0.25 μM; nitazoxanide, 10 μM; CGS-14943, 10 μM; and resveratrol 10 μM). Mean of four replicates shown, error bars represent standard deviation. For each set of bars, from left to right, results are shown for 2′OMe PS alone, then with febuxostat, lonafarnib, nitazoxanide, CGS-14943, and resveratrol.

FIG. 13 shows antisense oligo (ASO) activity (RLU) as a function of ASO (2′-MOE phosphorothioate, 2′-MOE PS) concentration (μM) with fixed oligo activity enhancer (OAE) concentration in an HeLa pLuc/705 assay (varied ASO concentration, enhancer concentrations fixed: febuxostat, 5 μM; lonafarnib, 0.25 μM; nitazoxanide, 10 μM; CGS-14943, 10 μM; and resveratrol 10 μM). Mean of four replicates shown, error bars represent standard deviation. For each set of bars, from left to right, results are shown for 2′-MOE PS alone, then with febuxostat, lonafarnib, nitazoxanide, CGS-14943, and resveratrol.

FIG. 14 illustrates cell viability as a function of febuxostat concentration (μM) in an alamarBlue cell viability assay. Mean of three replicates shown, error bars represent standard deviation.

FIG. 15 illustrates cell viability as a function of lonafarnib concentration (μM) in an alamarBlue cell viability assay. Mean of three replicates shown, error bars represent standard deviation.

FIG. 16 illustrates cell viability as a function of nitazoxanide concentration (μM) in an alamarBlue cell viability assay. Mean of three replicates shown, error bars represent standard deviation.

FIG. 17 illustrates cell viability as a function of CGS-15943 concentration (μM) in an alamarBlue cell viability assay. Mean of three replicates shown, error bars represent standard deviation.

FIG. 18 illustrates cell viability as a function of resveratrol concentration (μM) in an alamarBlue cell viability assay. Mean of three replicates shown, error bars represent standard deviation.

FIG. 19A compares antisense oligo (ASO) activity (RLU) as a function of a representative oligo activity enhancer (OAE) concentration (μM) (hydroxypropyl-beta-cyclodextrin febuxostat composition) in an HeLa pLuc/705 assay (varied OAE composition concentration with fixed ASO concentration: PPMO at 5 μM. Mean of three replicates shown, error bars represent standard deviation. FIG. 19B shows cell viability of the febuxostat composition as a function of febuxostat concentration (μM) in an alamarBlue cell viability assay (varied OAE composition concentration with fixed ASO concentration: PPMO at 5 M). Mean of three replicates shown, error bars represent standard deviation.

FIG. 20 compares SARS-CoV-2 virus titer over time in Calu-3 cell culture infection. OAE 1 is the febuxostat composition (10 M) and OAE 2 is lonafarnib (250 nM in DMSO). Means of duplicate samples shown for untreated (w/o), PPMO (ACE2-AUG) at 15 M and PPMO+OAE conditions. Single samples for scramble control PPMO and OAE only conditions.

FIG. 21 compares relative ACVR1 expression in fibrodysplasia ossificans progressiva (FOP) patient-derived fibroblasts under LNA or MOE gapmer treatment alone (MOE3 or LNA16), gapmer with Lipofectamine RNAiMAX (MOE3-Lipo or LNA16-Lipo), gapmer with febuxostat formulation (MOE3-OAE1 or LNA16-OAE1), or febuxostat formulation alone (OAE1). Three experiments represented: individual data points represent the mean of four wells from separate experiments; error bars represent standard deviation. Statistical results shown as follows: *=p<0.05, ***=p<0.001, ****=p<0.0001.

FIG. 22 compares relative DUX4 expression in facioscapulohumeral muscular dystrophy (FSHD) patient-derived muscle cells under treatment with LNA or MOE gapmers alone, gapmer with Lipofectamine RNAiMAX, or gapmer with febuxostat formulation. Mean of three experiments utilizing four replicates each is shown; error bars represent standard deviation. Statistical results shown as follows: *=p<0.05, **=p<0.01, ***=p<0.001, ****=p<0.0001.

### DETAILED DESCRIPTION

The present disclosure provides compounds and methods of using the compounds that solve the problem of inefficient intracellular (e.g., cytosolic) delivery of bioactive macromolecules. The compounds described herein that are useful for improving the intracellular (e.g., cytosolic) delivery of bioactive macromolecules are referred to herein as enhancer molecules.

In one aspect, the disclosure provides a method for improving the cytosolic delivery of bioactive macromolecules in a human subject, animal, or cells.

In another aspect, the disclosure provides a method for the cytosolic delivery of bioactive macromolecules to a subject.

In a further aspect, the disclosure provides a method for intracellular antisense oligo delivery to a subject. In certain embodiments, the method comprises contacting a cell with an antisense oligo, and an effective amount of an enhancer molecule. In other embodiments, the method comprises administering to a subject in need thereof an antisense oligo, and an effective amount of an enhancer molecule.

In another aspect, the disclosure provides a method for treating a disease or condition treatable by administering an antisense oligo. In certain of these embodiments, the method comprises administering to a subject in need thereof a therapeutically effective amount of an antisense oligo useful for treating a disease or condition and an effective amount of an enhancer molecule.

As used herein, the term “antisense oligo” (ASO) refers to nucleic acid oligomer or oligonucleotide analog (e.g., a single strand of synthetic DNA, RNA, PMO, 2′-O-substituted phosphorothioate, peptide nucleic acid, locked nucleic acid, etc.). The term “antisense” refers to a single stranded nucleic acid oligo complementary to an RNA with which it hybridizes, and thereby changes function of the RNA; note that the term “antisense” is not restricted to those sequences that are inverse complementary to sense (mRNA) strands, but include in the term “antisense” those oligo sequences which are complementary to any natural RNA, including circRNA, miRNA, lncRNA, piwiRNA, or other non-coding RNAs. Examples of alteration of RNA function are modulation of pre-messenger RNA splicing, inhibition of messenger RNA translation to protein, inhibition of ribosomal RNA function, blocking poly-A tailing signal sequences, blocking miRNA maturation and activity, blocking ribozyme activity, blocking RNA translocation sequences (adapter protein binding sites) and blocking other sites where proteins or protein-RNA complexes bind to RNA. Exemplary ASOs include those described herein.

In the above aspects and embodiments, the methods comprise administering an enhancer molecule in an amount effective to facilitate (e.g., increase) the cytosolic delivery of the bioactive macromolecule.

In the methods above, the enhancer molecule (OAE) is febuxostat, lonafarnib, nitazoxanide, CGS-15943, or resveratrol, or a pharmaceutically acceptable salt thereof. In the above methods, one or more of the enhancer molecules is administered to a subject in combination with one or more bioactive macromolecule. In certain embodiments, the enhancer molecule is administered to the subject in combination with the bioactive macromolecule (i.e., at the same time, for example, as in a mixture, simultaneous dosing). In other embodiments, the enhancer molecule is administered to the subject prior to or after administration of the bioactive macromolecule (i.e., sequential dosing).

As used herein, with regard to bioactive macromolecule delivery, the terms “intracellular” and “cytosolic” are used interchangeably.

In a further aspect of the disclosure, enhancer molecule compositions are provided.

In certain embodiments, the composition comprises a bioactive macromolecule and an enhancer molecule selected from the group consisting of febuxostat, lonafarnib, nitazoxanide, CGS-15943, and resveratrol, and pharmaceutically acceptable salts thereof. As described herein, these compositions can be used for improving the cytosolic delivery of a bioactive macromolecule in a subject (e.g., human, animal, and cells).

In other embodiments, the composition comprises an antisense oligo and an enhancer molecule selected from the group consisting of febuxostat, lonafarnib, nitazoxanide, CGS-15943, and resveratrol, and pharmaceutically acceptable salts thereof. These compositions can be used for intracellular antisense oligo delivery in a subject.

The chemical structures of the representative enhancer molecules (OAE) described herein are shown below.

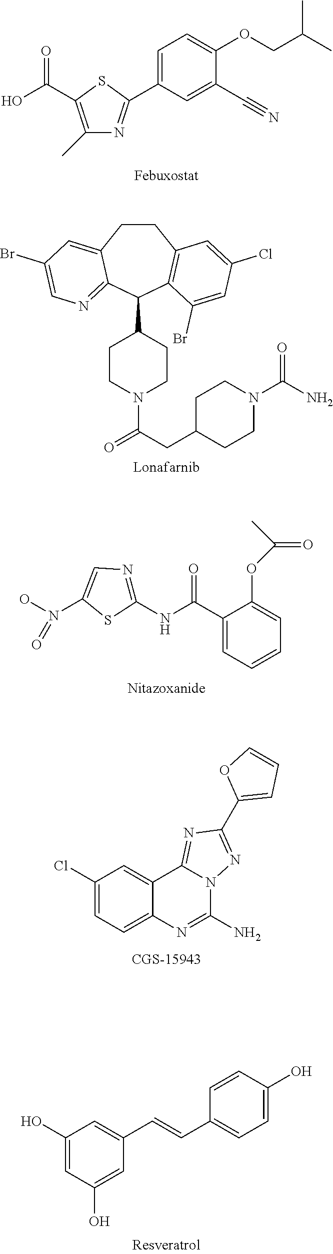

The enhancer molecules described here are used in the treatment of cells and in vivo treatment.

Febuxostat is used to significantly enhance the activity of ASOs and intracellular delivery of other macromolecules (RNA, siRNA, peptides or proteins) upon co-treatment of cells, simultaneously or sequentially, with febuxostat and the ASO or macromolecule, where febuxostat concentrations are at or within the range 0.25-20 μM. Following oral dosage with 240 mg febuxostat, the mean maximum plasma value in humans was 26.5 μM. This concentration lies well above the febuxostat concentrations found to have significant ASO enhancing activity in cell culture experiments. The 240 mg dose corresponds to a dosage of 4 mg/kg for a human of average weight 60 kg. Febuxostat is used to significantly enhance the activity of ASOs and intracellular delivery of other macromolecules (RNA, siRNA, peptides or proteins) upon co-administration, simultaneously or sequentially, of febuxostat and the ASO or macromolecule, where febuxostat dosage is within the range 0-40 mg/kg.

Lonafarnib is used to significantly enhance the activity of ASOs and intracellular delivery of other macromolecules (RNA, siRNA, peptides or proteins) upon co-treatment of cells, either simultaneously or sequentially, with lonafarnib and the ASO or macromolecule, where lonafarnib concentrations are at or within the range 0.1-20 μM. Lonafarnib has been used in humans at a dose of 300 mg, which corresponds to a dosage of 5 mg/kg with average human weight 60 kg and 166.7 mg/m² for a human of body surface area 1.8 m². Following oral dosage with 150 mg/m² lonafarnib, the mean maximum plasma value in human progeria patients was 4.13 M. This concentration lies well above the lonafarnib concentrations found to have significant ASO enhancing activity in cell culture experiments. Lonafarnib is used to significantly enhance the activity of ASOs and intracellular delivery of other macromolecules (RNA, siRNA, peptides or proteins) upon co-administration, either simultaneously or sequentially, of lonafarnib and the ASO or macromolecule, where lonafarnib dosage is within the range 0-20 mg/kg.

Nitazoxanide is used to significantly enhance the activity of ASOs and intracellular delivery of other macromolecules (RNA, siRNA, peptides or proteins) upon co-treatment of cells, either simultaneously or sequentially, with nitazoxanide and the ASO or macromolecule, where nitazoxanide concentrations are at or within the range 5-20 μM. Nitazoxanide has been used in humans at doses ranging to 4 g. This dose corresponds to a dosage of 66.7 mg/kg with average human weight 60 kg. Nitazoxanide has been shown to degrade very rapidly in human plasma to a related compound, tizoxanide, and thus pharmacokinetic studies in humans have quantified tizoxanide and its glucuronide metabolite, rather than nitazoxanide directly. Nitazoxanide is used to significantly enhance the activity of ASOs and intracellular delivery of other macromolecules (RNA, siRNA, peptides or proteins) upon co-administration, either simultaneously or sequentially, of nitazoxanide and the ASO or macromolecule, where nitazoxanide dosage is within the range 0-80 mg/kg.

CGS-15943 is used to significantly enhance the activity of ASOs and intracellular delivery of other macromolecules (RNA, siRNA, peptides or proteins) upon co-treatment of cells, either simultaneously or sequentially, with CGS-15943 and the ASO or macromolecule, where CGS-15943 concentrations are at or within the range 0.25-20 μM. Human pharmacokinetic data are unavailable for CGS-15943. In mice, intraperitoneal administration of CGS-15943 at 10 mg/kg has been used with no reported adverse effects. CGS-15943 is used to significantly enhance the activity of ASOs and intracellular delivery of other macromolecules (RNA, siRNA, peptides or proteins) upon co-administration, either simultaneously or sequentially, of CGS-15943 and the ASO or macromolecule, where CGS-15943 dosage is within the range 0-20 mg/kg.

Resveratrol is used to significantly enhance the activity of ASOs and intracellular delivery of other macromolecules (RNA, siRNA, peptides or proteins) upon co-treatment of cells, either simultaneously or sequentially, with resveratrol and the ASO or macromolecule, where resveratrol concentrations are at or within the range 1-20 μM. Human pharmacokinetic data indicate low plasma resveratrol concentrations following oral administration, with maximum plasma concentrations reaching 2.4 μM following a single oral dose of 5 g. This is below the optimal enhancing range identified herein in cell culture experiments. However, other routes of administration, for example, bypassing immediate hepatic metabolism, will increase resveratrol bioavailability. The 5 g dose corresponds to a dosage of 83.3 mg/kg with average human weight 60 kg. Resveratrol is used to significantly enhance the activity of ASOs and intracellular delivery of other macromolecules (RNA, siRNA, peptides or proteins) upon co-administration, either simultaneously or sequentially, of resveratrol and the ASO or macromolecule, where resveratrol dosage is within the range 0-100 mg/kg.

In the methods described herein, bioactive macromolecules are delivered (e.g., intracellular, cytosolic). In the compositions described herein, bioactive macromolecules are a component. As used herein, the term “bioactive macromolecule” refers to a macromolecule having advantageous bioactivity, and the term “macromolecule” refers to a polymer such as a poly(nucleic acid) (e.g., an oligonucleotide, such as a DNA or an RNA) or a poly(amino acid) (e.g., a polypeptide, protein, or functional protein fragment). Representative bioactive macromolecules include antisense oligos (ASO), RNAs, siRNAs, unmodified or modified nucleic acids, peptides, and proteins. In certain embodiments, the bioactive macromolecule is an antisense oligo.

Representative antisense oligo (ASOs) include phosphorodiamidate morpholino oligomers (PMOs), peptide-conjugated phosphorodiamidate morpholino oligomers (PPMOs), 2′-O-methyl phosphorothioate (2′-OMe PS) oligomers, 2′-O-methoxyethyl phosphorothioate (2′-MOE PS) oligomers, peptide nucleic acids (PNA), locked nucleic acids (LNA), LNA and 2′-OMe PS gapmers, LNA and 2′-MOE gapmers, gapmers of 2′-OMe PS and natural nucleic acids, and gapmers of 2′OMOE PS and natural nucleic acids.

The chemical structures of representative antisense oligos (ASOs) are shown below.

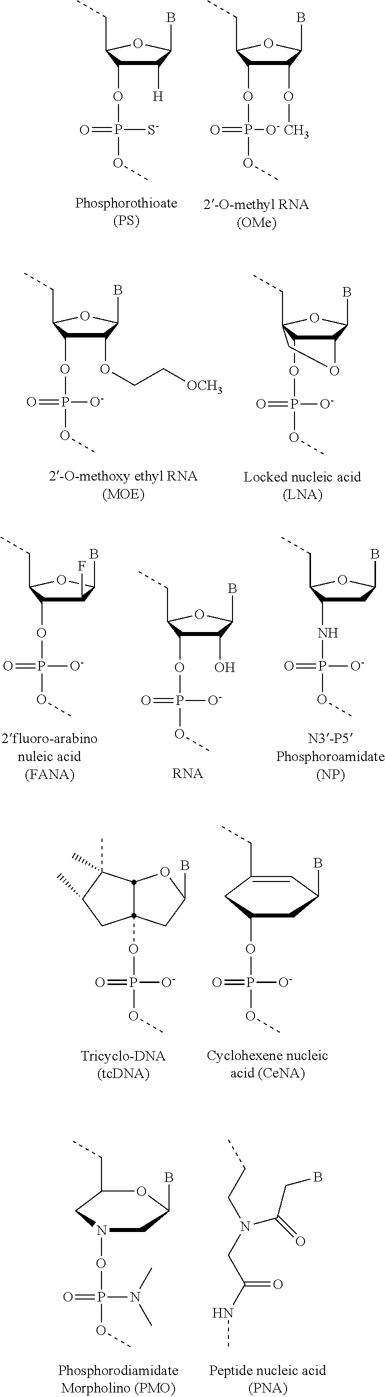

The following is a description of representative antisense oligo activity enhancers (OAEs) and their use in the methods for improving cytosolic delivery.

### Antisense Oligo Activity Enhancers

Antisense oligos (ASOs) are powerful tools for the control of gene expression which have been employed for numerous uses, including as therapeutic agents. ASO types include phosphorodiamidate morpholino oligomers (PMOs), peptide-conjugated phosphorodiamidate morpholino oligomers (PPMOs), 2′-O-methyl phosphorothioate (2′-OMe PS) oligomers, 2′-O-methoxyethyl phosphorothioate (2′-MOE PS) oligomers, peptide nucleic acids (PNA), locked nucleic acids (LNA), LNA and 2′-OMe PS gapmers, LNA and 2′-MOE gapmers, gapmers of 2′-OMe PS and natural nucleic acids, and gapmers of 2′OMOE PS and natural nucleic acids. While rational control of gene expression through targeting of native nucleic acids has been useful in many arenas, intracellular delivery remains an imposing bottleneck for all kinds of ASO technology. Generally, a large majority of ASO that enter a cell through endocytosis remain entrapped within the endosome and are thus ineffective for their intended use. Potentiating ASO activity through the use of non-covalent co-treatment agents holds great promise as a practical approach for overcoming this delivery challenge. Using high-throughput screening of compound libraries containing about 4000 compounds, the inventors identified five such agents, referred to here as oligo activity enhancers (OAEs or “enhancer molecules”), that increase ASO activity upon non-covalent co-treatment with ASOs. The activity of these enhancers was characterized and reproduced screening results. Screening was focused on FDA-approved drugs, with the rationale that such agents, if found to enhance oligo activity, would be relatively more practical to implement for clinical use than the non-FDA-approved compounds.

HeLa pLuc/705 Assay and Identification of OAEs Using High-Throughput Screening

The Hela pLuc/705 assay provides a positive readout of ASO's splice-modifying activity. Because messenger RNA (mRNA) splicing occurs in the cell's nucleus, the assay's output is indicative of the effectiveness of delivery of splice-modifying oligomers into the nucleus, which necessarily implies effective cytosolic delivery. The assay utilizes a genetically modified HeLa cell line that has been stably transfected with a gene containing coding sequence for a reporter enzyme (luciferase) and a mutated intron inserted within the coding sequence. The mutated intron cannot be properly excised resulting in an in-frame stop codon in the luciferase coding sequence, which prevents expression of functional luciferase. Two conditions must be met to produce the functional enzyme. First, an ASO must enter the cell nucleus to block the mutated splice site, thus restoring normal splicing and read through of the reporter message. The result is mRNA encoding functional reporter protein. Second, but equally important, the cells must be viable to carry out RNA processing and translation so that reporter protein activity is produced.

Correction through successful nuclear delivery of a splice-modifying ASO restores luciferase expression, which is then quantified by measurement of cell lysate luminescence with addition of luciferin substrate. This assay is a standard measure of intracellular ASO delivery within the field.

The HeLa pLuc/705 assay was miniaturized in order to allow it to be performed in 348-well cell culture plates, enabling the assay to be adapted to the high-throughput screening setting. The aim was to screen small molecules, with an emphasis on FDA-approved drugs, for ability to enhance ASO splice-modifying activity, which would be indicated by an increase in luminescence relative to cells treated with the ASO alone. In order to validate the approach, a positive control molecule was utilized which was already known to increase ASO activity when used alongside the ASO. Initially, a peptide intended for use in cell culture was used. Subsequently, febuxostat was used as a positive control.

A PPMO that is effective for luciferase splice correction within HeLa pLuc/705 cells was used to screen the compound libraries. The first screened compounds were the compounds in the Selleck Chemicals FDA-approved drug library for enhancing activity. This library contains 1430 compounds. Library compounds were dispensed to 10 μM in the plate wells. Compounds were identified as hits if treated cells displayed luminescence values above those attained under treatment with peptide control at 5 μM. Sixteen compounds met this criterion and were selected for secondary screening. These sixteen compounds were evaluated for enhancing performance over the concentration range 0.1-20 μM. This enabled validation of the primary screening results, as well as to evaluate relative performance of hits through observation of concentration-dependent activity. Hits were evaluated based on relative enhancing performance, and they were also qualitatively evaluated for suitability for future therapeutic use, considering the identity, indication and approval status of the compound. Two compounds emerged as strong performers: febuxostat and lonafarnib. Resveratrol, which is not an FDA-approved drug, was also found to be an OAE.

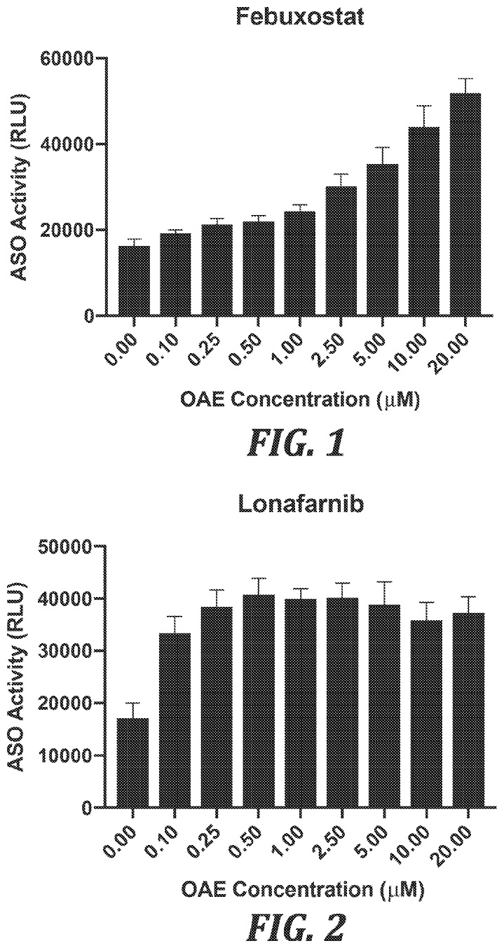

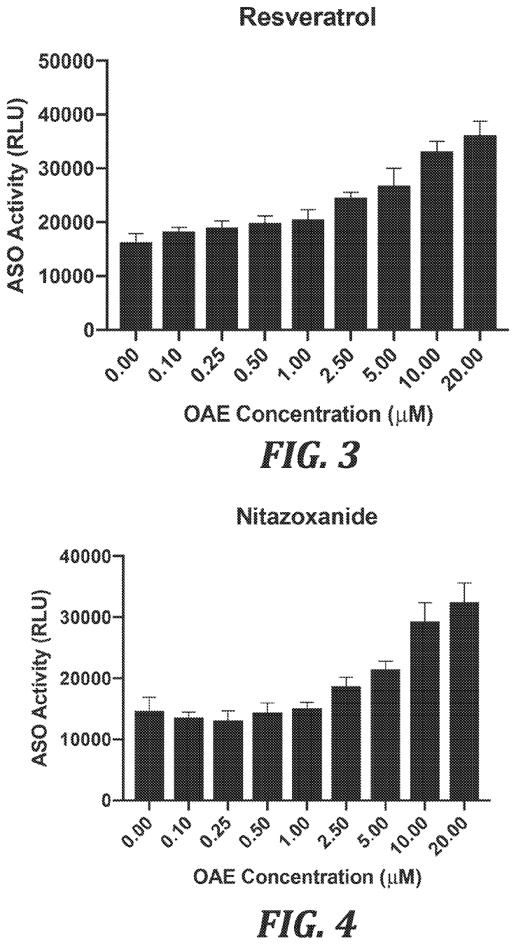

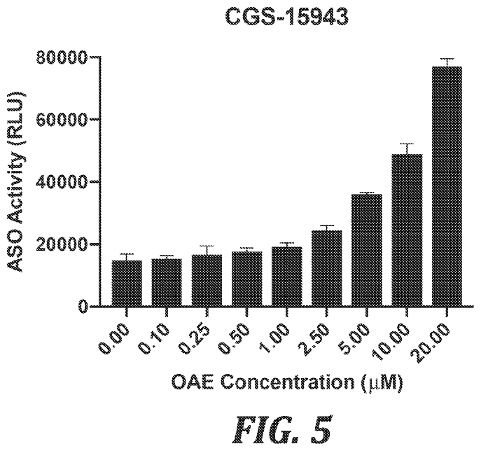

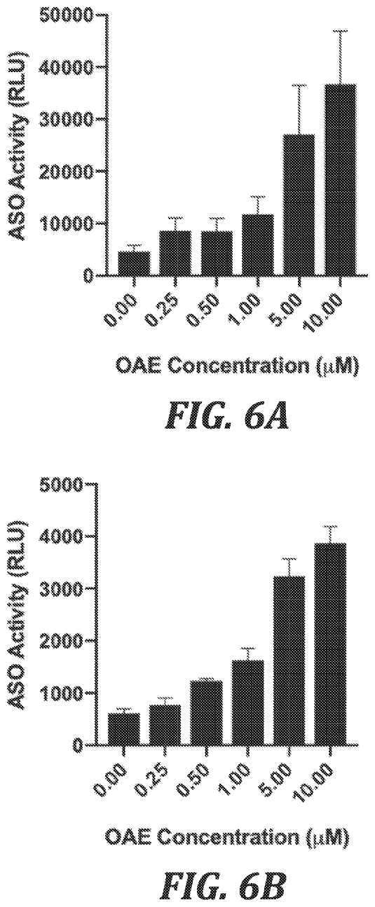

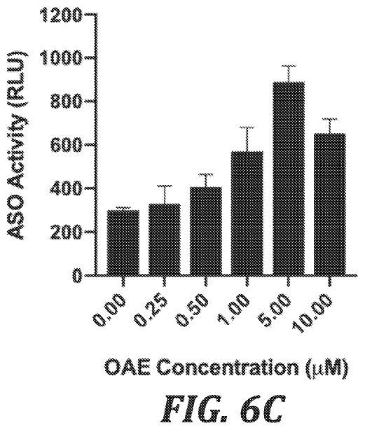

The compounds that were next screened were the compounds in the National Institutes of Health Clinical Collection, which contains 446 compounds that have been used in human clinical trials. Two compounds, nitazoxanide and CGS-15943, were found to provide significant enhancement of PPMO activity. A screen of the MicroSource Discovery Spectrum library did not yield further candidate OAEs, but febuxostat was identified as a strong hit during this screen, highlighting the reproducibility of our previous findings. Referring to FIGS. 1-5, at the conclusion of library screening, three candidates with prior FDA approval were identified: febuxostat, lonafarnib, and nitazoxanide. Two further candidates that are not FDA-approved drugs were also identified: CGS-15943 and resveratrol. CGS-15943 is a non-xanthine adenosine receptor antagonist that has shown promise as an anti-cancer agent. Resveratrol is a natural product that has been extensively researched within a wide range of disease contexts, including cancer, heart disease, metabolic and inflammatory conditions.

Demonstration of OAE Performance with a Range of ASO Types

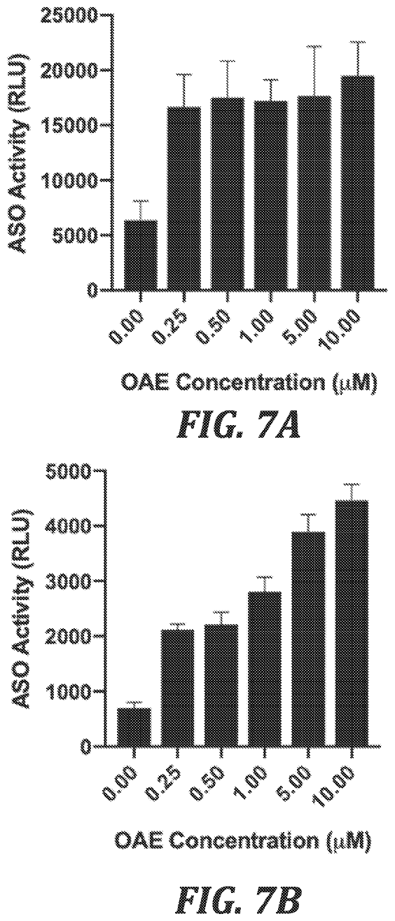

To explore the breadth of potential applications of the OAEs, the performance of febuxostat, lonafarnib, CGS-15943 and resveratrol were tested within the HeLa pLuc/705 assay using three distinct ASO structural types: PPMO, 2′-OMe PS and 2′-MOE PS. ASO concentrations were fixed for all conditions, while OAE concentrations were varied to demonstrate concentration-dependence of effects. Referring to FIGS. 6A-6C, febuxostat showed significant enhancement of PPMO activity (7.9-fold increase at 10 μM), 2′-OMe PS activity (6.4-fold increase at 10 μM), and 2′-MOE PS activity (3-fold increase at 5 μM), respectively.

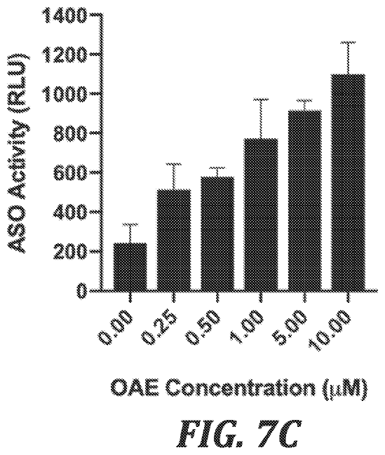

Referring to FIGS. 7A-7C, lonafarnib showed significant enhancement of the activity of each ASO type, displaying its unique strength: enhancement is marked within the nanomolar concentration range. At 250 nM, lonafarnib increased PPMO activity 2.6-fold, 2′-OMe PS activity 3-fold, and 2′-MOE PS activity 2.1-fold.

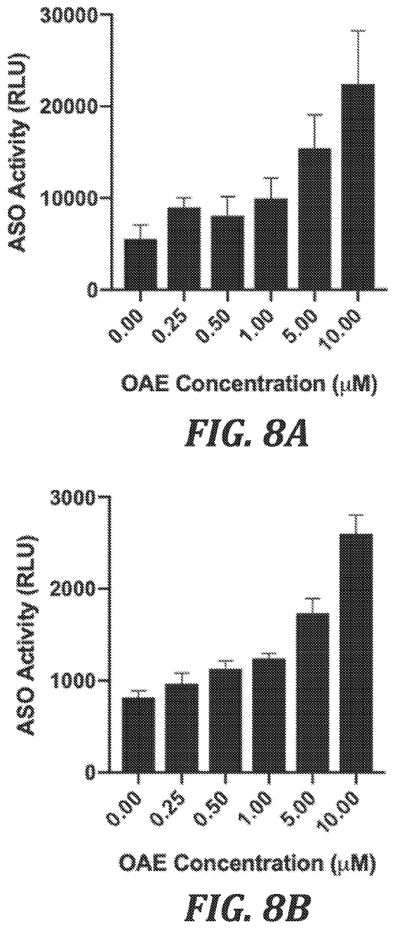

Referring to FIGS. 8A-8C, CGS-15943 showed significant enhancement of PPMO activity (4-fold at 10 μM), 2′-OMe PS activity (3.2-fold at 10 μM), and 2′-MOE PS activity (2.6-fold at 10 μM).

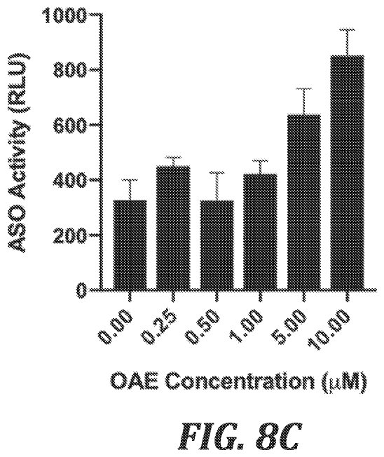

Referring to FIGS. 9A and 9B, resveratrol did not show enhancement of 2′-MOE PS activity, but caused a 2.1-fold increase in PPMO activity and a 1.5-fold increase in 2′-OMe PS activity at 10 μM.

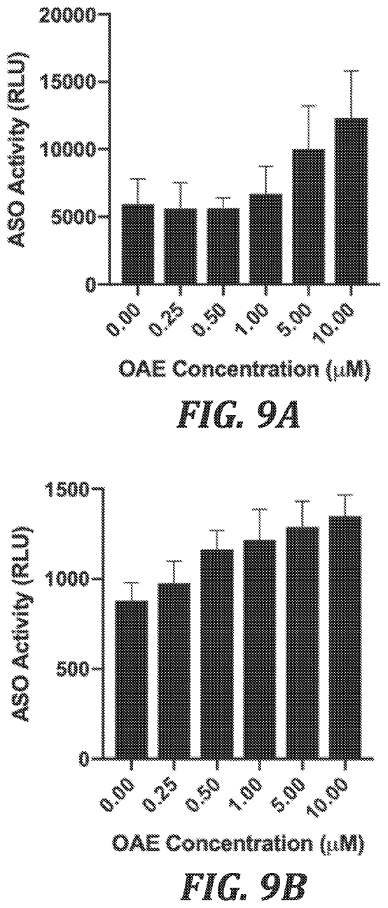

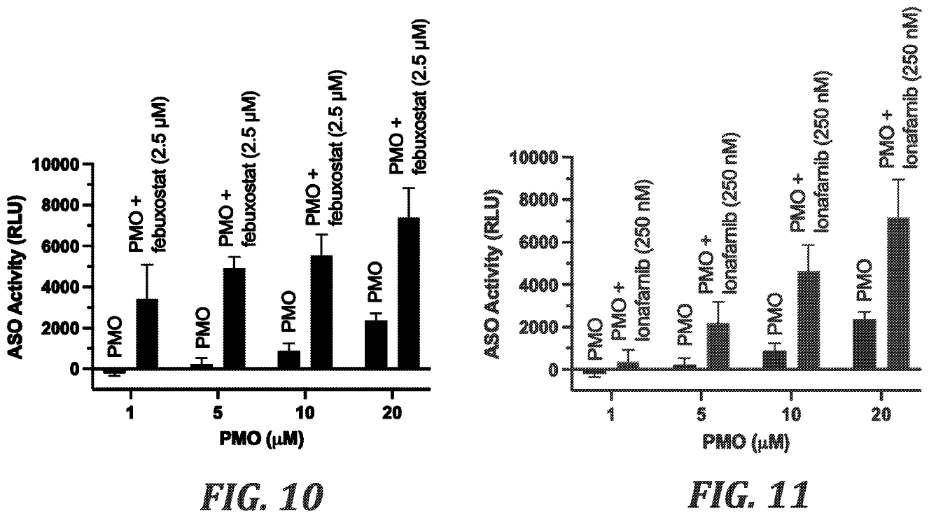

Referring to FIGS. 10 and 11, febuxostat and lonafarnib were found to enhance the activity of unmodified PMOs, which represents another ASO structure type. These results were generated within the manual format (96 wells) of the HeLa pLuc/705 assay.

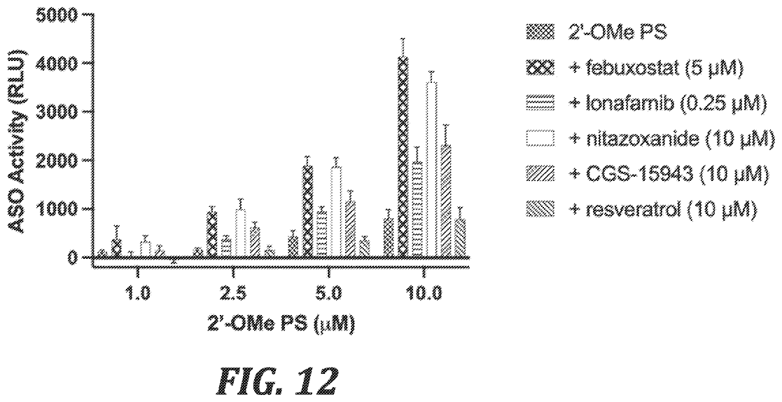

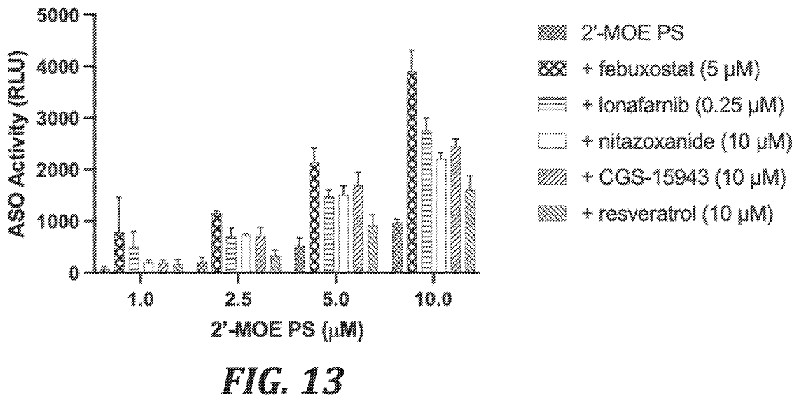

Referring to FIGS. 12 and 13, assays utilizing fixed OAE concentrations with varied 2′-OMe PS and 2′-MOE PS ASO concentrations were also performed. Resveratrol failed to enhance activity of the 2′-OMe PS ASO, but did show enhancement of the 2′-MOE PS ASO in this format. Low signal magnitude with phosphorothioate ASO treatment combined with relatively weak enhancement by resveratrol are likely the cause of apparent inconsistencies for resveratrol enhancement of phosphorothioate oligo activity.

### OAE Cytotoxicity

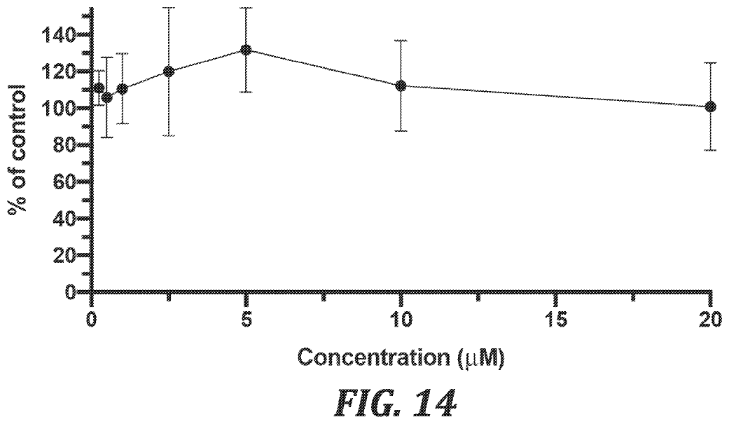

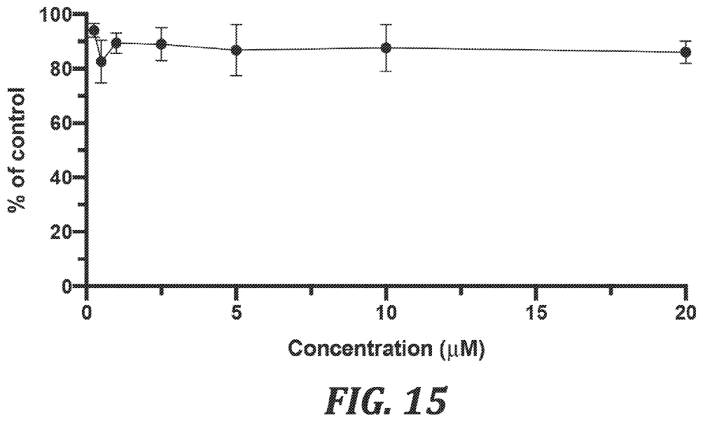

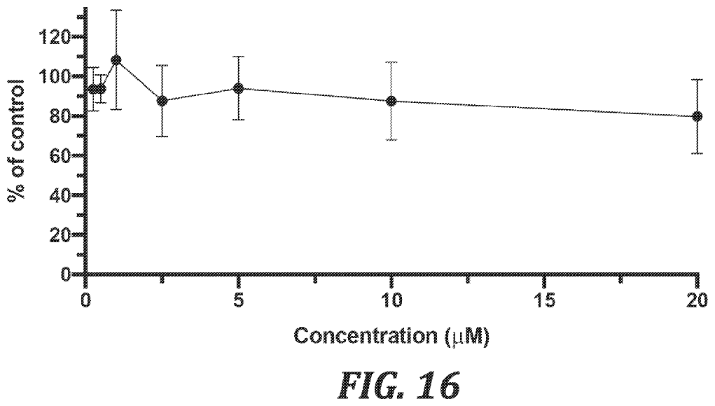

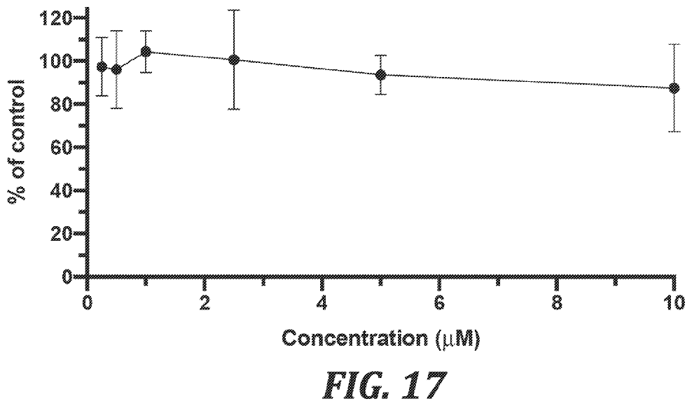

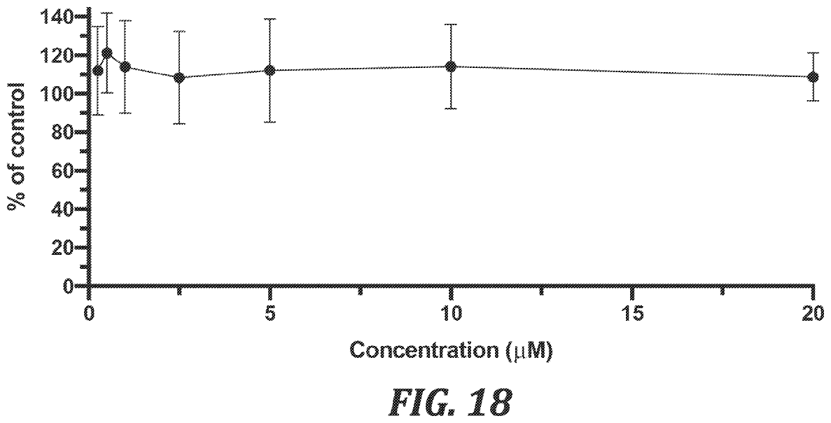

Referring to FIGS. 14-18, using the alamarBlue assay, which indicates cell viability based on relative reducing capacity, the cytotoxicity of representative OAEs was evaluated. None of the OAEs demonstrated significant cytotoxicity within tested enhancing concentration range.

### Representative OAE Compositions

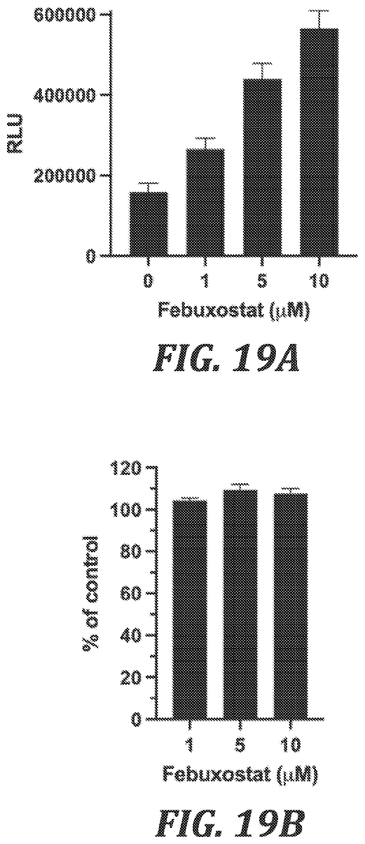

A representative water-soluble febuxostat formulation was developed for use in both cellular and in vivo applications. See Example 1. The formulation included a lyophilized powder composed of febuxostat and the excipient hydroxypropyl-beta-cyclodextrin. This formulation demonstrated activity within the HeLa pLuc/705 assay comparable to that shown by febuxostat in DMSO solution and did not show cytotoxic effects when used in combination with PPMO as indicated by the alamarBlue cytotoxicity assay. See FIGS. 19A and 19B. Febuxostat was combined with hydroxypropyl-beta-cyclodextrin and lyophilized following dissolution of febuxostat to produce a composition employed for assays and methods described herein. Alternatively, febuxostat may be combined with excipients such as alpha-cyclodextrin or its derivatives, beta-cyclodextrin or its derivatives other than hydroxypropyl-beta-cyclodextrin, gamma-cyclodextrin or its derivatives, polyethylene glycol with molecular weight 300-1500 g/mol, poloxamer 124, poloxamer 182, poloxamer 188, poloxamer 331, poloxamer 407, polysorbate 20, polysorbate 80 or sodium bicarbonate to produce a composition employed for the assays and methods described herein.

### Knockdown of Angiotensin-Converting Enzyme 2 (ACE2) in SARS-CoV-2-Infected Human Lung Cells

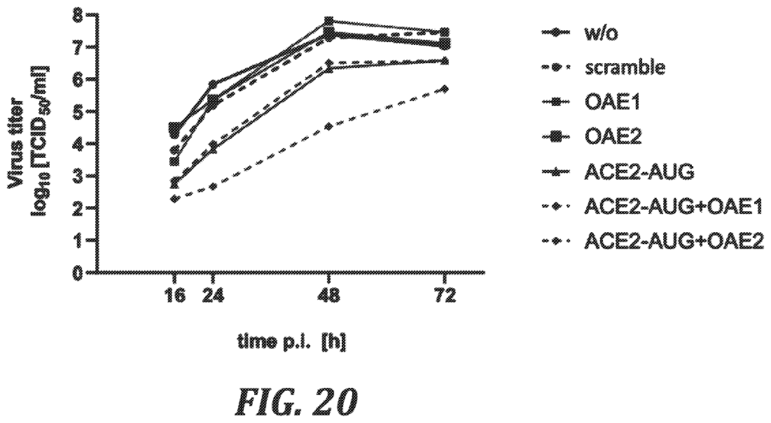

The representative febuxostat composition was evaluated in a PPMO-mediated knockdown of angiotensin-converting enzyme 2 (ACE2) in SARS-CoV-2-infected human lung cells as described in Example 2. Referring to FIG. 20, use of the febuxostat composition alongside a PPMO designed to block translation of ACE2 (ACE2-AUG) within human lung cells resulted in lower SARS-CoV-2 virus titers than treatment using the PPMO alone.

### Fibrodysplasia Ossificans Progressiva (FOP)

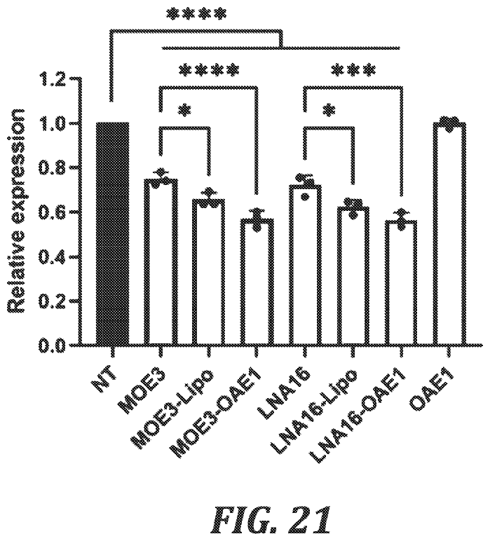

The representative febuxostat formulation was used to enhance the activity of LNA and MOE gapmers targeting expression of ACVR1 within FOP patient-derived fibroblasts as described in Example 3. FIG. 21 compares relative ACVR1 expression in fibrodysplasia ossificans progressiva (FOP) patient-derived fibroblasts under LNA or MOE gapmer treatment alone (MOE3 or LNA16), gapmer with Lipofectamine RNAiMAX (MOE3-Lipo or LNA-Lipo), gapmer with febuxostat formulation (MOE3-OAE1 or LNA16-OAE1), or febuxostat formulation alone (OAE1).

### Facioscapulohumeral Muscular Dystrophy (FSHD)

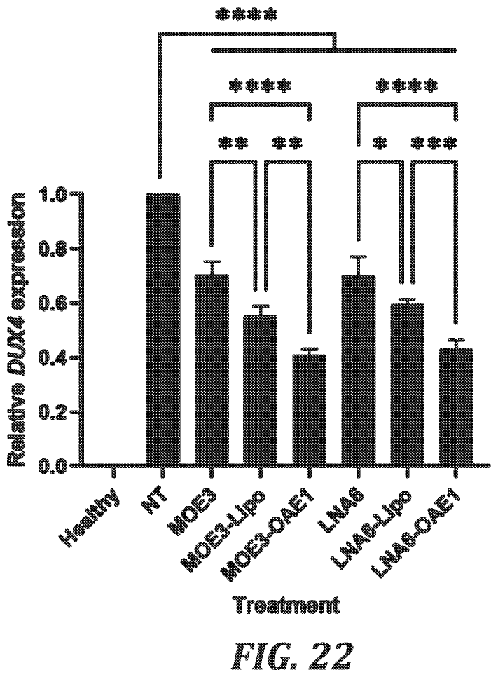

The representative febuxostat formulation was used to enhance the activity of LNA and MOE gapmers targeting expression of DUX4 within FSHD patient-derived muscle cells as described in Example 4. FIG. 22 compares relative DUX4 expression in facioscapulohumeral muscular dystrophy (FSHD) patient-derived muscle cells under treatment with LNA or MOE gapmers alone, gapmer with Lipofectamine RNAiMAX, or gapmer with febuxostat formulation.

### Methods of OAE Use

ASOs and other macromolecules such as RNAs, proteins and peptides are rapidly expanding in their use as therapeutic agents and research tools. Numerous therapies employing these macromolecules are currently in use for a wide range of indications and optimizing delivery of these new therapeutic tools is central to progress in this area. PPMOs, the ASO type for which OAEs were first found to provide enhancing activity, have been found effective within numerous applications, including as antiviral agents, agents for targeting bacteria and eukaryotic parasites, agents for addressing genetic diseases including Duchenne muscular dystrophy, spinal muscular atrophy, Hutchinson-Gilford progeria syndrome and cystic fibrosis, and as an anti-cancer agent. Important routes of administration are diverse, including systemic delivery by intravenous injection, intrathecal injection for delivery to the central nervous system, transdermal, ocular and oral routes. The OAEs described herein represent a valuable and novel general strategy for facilitating intracellular delivery of therapeutic macromolecules and enhancing activity over a range of applications due to the general nature of OAE activity. Additionally, the ability to improve intracellular delivery of macromolecules used for biological research holds great value.

The following examples are provided for the purpose of illustrating, not limiting the present disclosure.

### EXAMPLES

### Methods

HeLa pLuc/705 Assay

HeLa pLuc/705 cells were seeded into 384-well plates (50 L well volume) and incubated for 24 hours. At 24 hours, DMSO solutions of OAEs were added to subconfluent cells using an HP D300 Digital Dispenser, followed by addition of a testing ASO or DMSO. Cells were incubated for 24 hours. At 24 hours treatment time, growth medium was removed from the cells and 100 μL of a lysis buffer/luciferin solution was added to each well. Luminescence was measured using a BioTek plate reader 5 minutes following addition of the lysis buffer/luciferin solution. Background luminescence values (generated using OAE only/DMSO-matched controls) were subtracted from experimental values. These methods apply to the commonly employed format for the assay using the HP D300 Digital Dispenser, with slight variations used for the high-throughput and manual formats.

alamarBlue Assay

HeLa pLuc/705 cells were seeded into 96-well plates (200 L well volume) and incubated for 24 hours. At 24 hours, with cells subconfluent, growth medium was removed and replaced with fresh growth medium. DMSO solutions of OAEs were added and cells were incubated. 24 hours following treatment, OAE-containing medium was removed and replaced with fresh growth medium. Cells were incubated for 22 hours, and then 20 L alamarBlue reagent was added to each well. Two hours following reagent addition, fluorescence was measured at 530/590 nm using a Tecan plate reader. Fluorescence background values were subtracted and fluorescence values (representing cell viability) were expressed as percent of vehicle-treated control. The alamarBlue assay was performed with slight modifications for the experiment depicted in FIGS. 19A and 19B, with reduced well volume and no post-treatment incubation prior to the assay endpoint.

### Example 1

### Representative Febuxostat Composition

In this example, the preparation of a representative febuxostat composition is described.

A 45% w/v hydroxypropyl-beta-cyclodextrin solution was prepared in deionized water, to which was added excess solid febuxostat. Febuxostat was then dissolved through extensive mechanical agitation, and the mixture was subjected to centrifugation at 10 000×g for 5 minutes. The supernatant was then filtered using a 0.2 μm syringe filter and febuxostat content was quantified through use of a febuxostat concentration standard curve utilizing the febuxostat absorbance maximum at 315 nm. The solution was then diluted to approximately 1 mM in sterile deionized water, again quantified and lyophilized in small volumes corresponding to 100 nmole febuxostat.

### Example 2

### PPMO-Mediated Knockdown of Angiotensin-Converting Enzyme 2 (ACE2) in SARS-CoV-2-Infected Human Lung Cells

In this example, the effectiveness of the representative febuxostat composition, prepared as described in Example 1, in the PPMO-mediated knockdown of angiotensin-converting enzyme 2 (ACE2) in SARS-CoV-2-infected human lung cells is described.

Calu-3 cells were cultured in DMEM/F12 containing 10% fetal calf serum (FCS), glutamine and penicillin/streptomycin, then pre-treated for 24 hours using ACE2-translation-targeting PPMO at 15 M, the febuxostat composition (prepared as described above) at 10 M, lonafarnib (from DMSO solution) at 250 nM or combinations thereof in DMEM containing glutamine, penicillin/streptomycin and 0.1% bovine serum albumin (BSA). Treatment media were removed and cells were infected with SARS-CoV-2 Munich 929 isolate at a multiplicity of infection (MOI) of 0.00005. Virus titers were evaluated using a TCID50 assay. Results are summarized in FIG. 20.

### Example 3

### Fibrodysplasia Ossificans Progressiva (FOP)

The representative febuxostat formulation prepared as described in Example 1 was used to enhance the activity of LNA and MOE gapmers targeting expression of ACVR1 within FOP patient-derived fibroblasts.

Human fibroblasts from an individual affected by FOP carrying the heterozygous ACVR1^{R206H} mutation were cultured in DMEM/F12 medium with 10% fetal bovine serum and penicillin-streptomycin. Cells were seeded into 6-well plates, and the cells were treated at 60-70% confluency using 10 nM allele-specific gapmers alone or with 0.3% Lipofectamine RNAiMAX or febuxostat formulation. At 48 hours exposure, RNA was extracted using the standard TRIzol-based technique. cDNA was synthesized using oligo(dT)12-18 and SuperScript IV reverse transcriptase, then used for qPCR reactions employing TaqMan Fast Advanced Master Mix, using probes targeting ACVR1 (FAM) and RPS18 (VIC). Expression of ACVR1 was normalized to RPS18, then qPCR data were processed using a standard 2^{-ΔΔCt} algorithm and statistically analyzed using a one-way ANOVA with posthoc Tukey's comparison test. Results are summarized in FIG. 21.

### Example 4

### Facioscapulohumeral Muscular Dystrophy (FSHD)

The representative febuxostat formulation prepared as described in Example 1 was used to enhance the activity of LNA and MOE gapmers targeting expression of DUX4 within FSHD patient-derived muscle cells.

Immortalized myoblasts from an FSHD-affected patient and an unaffected individual were used for the experiments. Cells were cultured in a medium containing 15% fetal bovine serum, 2.5 ng/mL human hepatocyte growth factor, 10 ng/mL human fibroblast growth factor and 0.055 mg/mL dexamethasone. Basal medium was prepared by supplementing 20% Medium 199, 0.03 mg/mL zinc sulphate, 1.4 mg/mL vitamin B12 and penicillin-streptomycin. Differentiation medium was used to generate myotubes, and it contained 15% KnockOut Serum Replacement, 10 mg/mL insulin and 100 mg/mL human apo-transferrin. Cells were seeded into gelatin-coated 6-well plates and switched to differentiation medium when approximately 75% confluent. Treatment was performed 13 days post-differentiation using 10 nM DUX4-targeting gapmers with or without 10 M febuxostat formulation or 0.3% Lipofectamine RNAiMAX. Cells were harvested the next day and RNA extraction was performed using QiaShredder and RNeasy Mini Kit. cDNA was synthesized using oligo(dT)12-18 and SuperScript IV Reverse Transcriptase. qPCR was performed using SsoAdvanced Universal SYBR Green Supermix with primers specific to DUX4 and GAPDH. DUX4 expression was normalized to GAPDH, and data were processed using a standard 2^{-ΔΔCt} method. Statistical analysis utilized a one-way ANOVA with posthoc Tukey's comparison test. Results are summarized in FIG. 22.

While illustrative embodiments have been illustrated and described, it will be appreciated that various changes can be made therein without departing from the spirit and scope of the invention.

1. A method for cytosolic delivery of a bioactive macromolecule in a subject, comprising administering to a subject in need thereof a therapeutically effective amount of a bioactive macromolecule and an effective amount of an enhancer molecule selected from the group consisting of febuxostat, lonafarnib, nitazoxanide, CGS-15943, and resveratrol and pharmaceutically acceptable salts thereof.

2. The method of claim 1, wherein the bioactive macromolecule is selected from the group consisting of antisense oligos, RNAs, siRNAs, peptides, and proteins.

3. The method of claim 1, wherein the bioactive macromolecule is an antisense oligo.

4. The method of claim 1, wherein the bioactive macromolecule is an antisense oligo selected from phosphorodiamidate morpholino oligomers (PMOs), peptide-conjugated phosphorodiamidate morpholino oligomers (PPMOs), 2′-O-methyl phosphorothioate (2′OMe PS) oligomers, 2′-O-methoxyethyl phosphorothioate (2′-MOE PS) oligomers, peptide nucleic acids (PNA), locked nucleic acids (LNA), LNA and 2′OMe PS gapmers, LNA and 2′MOE gapmers, gapmers of 2′OMe PS and natural nucleic acids, gapmers of 2′OMOE PS and natural nucleic acids, or unmodified or modified nucleic acids.

5. The method of claim 1, wherein the bioactive macromolecule is an antisense oligo selected from phosphorodiamidate morpholino oligomers (PMOs), peptide-conjugated phosphorodiamidate morpholino oligomers (PPMOs), 2′-O-methyl phosphorothioate (2′OMe PS) oligomers, or 2′-O-methoxyethyl phosphorothioate (2′-MOE PS) oligomers.

6-8. (canceled)

9. A composition, comprising a bioactive macromolecule and an enhancer molecule selected from the group consisting of febuxostat, lonafarnib, nitazoxanide, CGS-15943, and resveratrol, and pharmaceutically acceptable salts thereof.

10. The composition of claim 9, wherein the bioactive macromolecule is selected from the group consisting of antisense oligos, RNAs, siRNAs, peptides, and proteins.

11. The composition of claim 9, wherein the bioactive macromolecule is an antisense oligo.

12. The composition of claim 9, wherein the bioactive macromolecule is an antisense oligo selected from phosphorodiamidate morpholino oligomers (PMOs), peptide-conjugated phosphorodiamidate morpholino oligomers (PPMOs), 2′-O-methyl phosphorothioate (2′OMe PS) oligomers, 2′-O-methoxyethyl phosphorothioate (2′-MOE PS) oligomers, peptide nucleic acids (PNA), locked nucleic acids (LNA), LNA and 2′OMe PS gapmers, LNA and 2′MOE gapmers, gapmers of 2′OMe PS and natural nucleic acids, gapmers of 2′OMOE PS and natural nucleic acids, or unmodified or modified nucleic acids.

13. The composition of claim 9, wherein the bioactive macromolecule is selected from phosphorodiamidate morpholino oligomers (PMOs), peptide-conjugated phosphorodiamidate morpholino oligomers (PPMOs), 2′-O-methyl phosphorothioate (2′OMe PS) oligomers, or 2′-O-methoxyethyl phosphorothioate (2′-MOE PS) oligomers.

14-16. (canceled)

17. A method for treating a disease or condition treatable by administering a bioactive macromolecule, comprising administering to a subject in need thereof a therapeutically effective amount of a bioactive macromolecule useful in treating a disease or condition and an effective amount of an enhancer molecule.

18. The method of claim 17, wherein the enhancer molecule is selected from a group consisting of febuxostat, lonafarnib, nitazoxanide, CGS-15943, and resveratrol, and pharmaceutically acceptable salts thereof.

19-23. (canceled)

24. The method of claim 17, wherein the bioactive macromolecule is selected from the group consisting of antisense oligos, RNAs, siRNAs, peptides, and proteins.

25. The method of claim 17, wherein the bioactive macromolecule is an antisense oligo.

26. The method of claim 17, wherein the bioactive macromolecule is an antisense oligo selected from phosphorodiamidate morpholino oligomers (PMOs), peptide-conjugated phosphorodiamidate morpholino oligomers (PPMOs), 2′-O-methyl phosphorothioate (2′OMe PS) oligomers, 2′-O-methoxyethyl phosphorothioate (2′-MOE PS) oligomers, peptide nucleic acids (PNA), locked nucleic acids (LNA), LNA and 2′OMe PS gapmers, LNA and 2′MOE gapmers, gapmers of 2′OMe PS and natural nucleic acids, gapmers of 2′OMOE PS and natural nucleic acids, or unmodified or modified nucleic acids.

27. The method of claim 17, wherein the bioactive macromolecule is selected from phosphorodiamidate morpholino oligomers (PMOs), peptide-conjugated phosphorodiamidate morpholino oligomers (PPMOs), 2′-O-methyl phosphorothioate (2′OMe PS) oligomers, or 2′-O-methoxyethyl phosphorothioate (2′-MOE PS) oligomers.
